(19) United States
(12) Patent Application Publication
(10) Pub. No.: US 2026/0207072 A1
(43) Pub. Date: Jul. 23, 2026

(54) RADAR-BASED NON-CONTACT ARRHYTHMIA DETECTION METHOD AND DEVICE

(71) Applicants: SEOUL NATIONAL UNIVERSITY HOSPITAL, Seoul (KR); KOREA ADVANCED INSTITUTE OF SCIENCE AND TECHNOLOGY, Daejeon (KR)

(72) Inventors: Si-Hyuck KANG, Seoul (KR); Insik SHIN, Daejeon (KR)

(21) Appl. No.: 19/116,348

(22) PCT Filed: Sep. 27, 2023
(86) PCT No.: PCT/KR2023/015043

§ 371 Date: Apr. 14, 2026

(30) Foreign Application Priority Data
Sep. 30, 2022 (KR) 10-2022-0125497
Sep. 26, 2023 (KR) 10-2023-0129639

(51) Int. Cl. A61B 5/05; A61B 5/00

(52) U.S. Cl. CPC A61B 5/05; A61B 5/7264; A61B 5/7271

(57) ABSTRACT

An arrhythmia detection device and method according to one embodiment are disclosed. The arrhythmia detection device according to one embodiment comprises: one or more processors; and a memory that stores one or more programs executed by the one or more processors, wherein the one or more processors acquire raw data of ultra-wideband radar signals of a certain period reflected from a subject, generate a first trajectory matrix on the basis of time series data converted from the raw data, decompose the first trajectory matrix to generate a second trajectory matrix, convert the second trajectory matrix to extract a cardiac signal, and if a specific wavelength interval in the cardiac signal is greater than or equal to a preset value, classify the subject as an arrhythmia patient.

### TECHNICAL FIELD

The disclosed embodiments relate to an ultra-wideband radar-based non-contact arrhythmia detection technology.

### CROSS-REFERENCE TO RELATED APPLICATION

This application claims priority to Korean Provisional Patent Application No. 10-2022-0125497, filed on Sep. 30, 2022, and Korean Patent Application No. 10-2023-0129639, filed on Sep. 26, 2023, the disclosures of which are incorporated herein in their entirety.

### BACKGROUND

An arrhythmia is a serious disease that is considered a major cause of sudden cardiac death and stroke and requires rapid examination. The arrhythmia is diagnosed using electrocardiogram (ECG) and ultrasound.

However, the ECG may limit movement of a patient due to electrodes attached to the patient and may cause contact dermatitis. The ultrasound is an expensive piece of equipment that must be handled by a skilled medical professional.

In order to solve the above problems, some studies have incorporated ultra-wideband (UWB) technology into the arrhythmia detection method. However, some studies estimate arrhythmias by measuring the number of heart beats or the time difference between heart beats.

That is, current studies have only adopted a modified simplification scheme to incorporate UWB technology beyond the traditional scheme. Traditionally, this is because arrhythmias are detected by analyzing an R peak interval, the P wave characteristics, the PR interval, and the QRS interval.

Since the electrical properties of the heart must be considered for the characteristics of some ECG waveforms, ECG measurements are inevitable. However, there is no relevant study on the R peak interval, although it can be detected via UWB technology.

That is, despite the existing studies, there is still a need for a new arrhythmia detection method that can overcome the disadvantages of the measurement device while following the traditional arrhythmia detection method.

### DETAILED DESCRIPTION OF THE INVENTION

### Technical Problem

The disclosed embodiments are intended to provide a method and a device for radar-based non-contact arrhythmia detection.

### Solution to Problem

An arrhythmia detection device according to an embodiment includes one or more processors, and a memory storing one or more programs executed by the one or more processors, wherein the one or more processors acquire raw data of ultra-wideband radar signals of a certain period reflected from a subject, generate a first trajectory matrix based on time series data converted from the raw data, decompose the first trajectory matrix to generate a second trajectory matrix, convert the second trajectory matrix to extract a cardiac signal, and classify the subject as an arrhythmia patient if a specific wavelength interval in the cardiac signal is greater than or equal to a preset value.

The one or more processors may identify the position of the subject's chest wall based on a maximum dynamic variation included in the raw data.

The one or more processors may generate the first trajectory matrix by arranging the time series data as components of the first trajectory matrix based on Equation 1 below, such that components of the remaining diagonals perpendicular to the main diagonal of the first trajectory matrix are all the same:

$$H = ( \begin{matrix}{x}_{1} & {x}_{2} & ⋯ & {x}_{n} \\ {x}_{2} & {x}_{3} & ⋯ & {x}_{n + 1} \\ ⋮ & ⋮ & ⋱ & ⋮ \\ {x}_{m} & {x}_{m - 1} & ⋯ & {x}_{L}\end{matrix} ) \qquad [ Equation 1 ]$$

H is the first trajectory matrix, xₐ is the a^{th} time series data, L is the length of the time series data, n and m are arbitrary natural numbers, provided that n=L−m+1, and 2≤m≤n.

The one or more processors may calculate a covariance matrix of the first trajectory matrix based on Equation 2 below:

$$C = \frac{1}{n} {HH}^{T} \qquad [ Equation 2 ]$$

C is the covariance matrix, H is the first trajectory matrix, H^{T} is the transpose of the first trajectory matrix, and n is the number of the time series data.

The one or more processors may decompose the first trajectory matrix into a plurality of second decomposition matrices based on a plurality of first decomposition matrices corresponding to the covariance matrix, which are obtained by performing the singular value decomposition (SVD) on the covariance matrix of the first trajectory matrix.

The one or more processors may calculate the plurality of second decomposition matrices using the S matrix, the U matrix, and the V^{T} matrix included in the plurality of first decomposition matrices, based on Equation 3 below:

$${H}_{i} = \sqrt{{λ}_{i}} {U}_{i} {V}_{i}^{T} \qquad [ Equation 3 ]$$

Hᵢ is the i^{th} second decomposition matrix of the plurality of second decomposition matrices, λ is the i^{th} eigenvalue of the covariance matrix, Uᵢ is the i^{th} column vector of the U matrix, and

$${V}_{i}^{T}$$

is the i^{th} row vector of the V matrix.

The one or more processors may select some matrices from among the plurality of second decomposition matrices by a preset number in order of the magnitude of the cardiac signal, filter the matrices other than a preset frequency band such that the cardiac signal corresponding to the matrices satisfies the frequency band, and generate the second trajectory matrix based on the remaining matrix remaining after filtering among the matrices.

The one or more processors may reconstruct the cardiac signal extracted from the second trajectory matrix based on Equation 4 below:

$$\hat{x} ( k ) = \{ \begin{matrix}{\sum }_{i = 1}^{k} \frac{1}{k} {\hat{x}}_{i , k + 1 - i} & ( 1 \leq k < m ) \\ {\sum }_{i = 1}^{m} \frac{1}{m} {\hat{x}}_{i , k + 1 - i} & ( m \leq k < n ) \\ {\sum }_{i = k - m + 1}^{m} \frac{1}{m + n - k} {\hat{x}}_{i , k + 1 - i} & ( n \leq k \leq m + n - 1 )\end{matrix} \qquad [ Equation 4 ]$$

\hat{x}(k) is the reconstructed k^{th} cardiac signal, k is the number of cardiac signals, and m and n are the number of rows or columns of the trajectory matrix.

The one or more processors may classify the subject as an arrhythmia patient if an R peak interval in the cardiac signal is greater than or equal to a preset value.

The arrhythmia detection method according to an embodiment is a method performed by an arrhythmia detection device including one or more processors and a memory storing one or more programs executed by the one or more processors, wherein the method includes obtaining raw data of ultra-wideband radar signals of a certain period reflected from a subject, generating a first trajectory matrix based on time series data converted from the raw data, decomposing the first trajectory matrix to generate a second trajectory matrix, converting the second trajectory matrix to extract a cardiac signal, and if a specific wavelength interval in the cardiac signal is greater than or equal to a preset value, classifying the subject as an arrhythmia patient.

The method may further include identifying the position of the subject's chest wall based on a maximum dynamic variation included in the raw data.

The generating of the first trajectory matrix may include generating the first trajectory matrix by arranging the time series data as components of the first trajectory matrix based on Equation 1 below such that components of the remaining diagonals perpendicular to the main diagonal of the first trajectory matrix are all the same:

$$H = ( \begin{matrix}{x}_{1} & {x}_{2} & ⋯ & {x}_{n} \\ {x}_{2} & {x}_{3} & ⋯ & {x}_{n + 1} \\ ⋮ & ⋮ & ⋱ & ⋮ \\ {x}_{m} & {x}_{m - 1} & ⋯ & {x}_{L}\end{matrix} ) \qquad [ Equation 1 ]$$

H is the first trajectory matrix, xₐ is the a^{th} time series data, L is the length of the time series data, n and m are arbitrary natural numbers, provided that n=L−m+1, and 2≤m≤n.

The generating of the second trajectory matrix may include calculating the covariance matrix of the first trajectory matrix based on Equation 2 below:

$$C = \frac{1}{n} {HH}^{T} \qquad [ Equation 2 ]$$

C is the covariance matrix, H is the first trajectory matrix, H^{T} is the transpose of the first trajectory matrix, and n is the number of the time series data.

The generating of the second trajectory matrix may include decomposing the first trajectory matrix into a plurality of second decomposition matrices based on a plurality of first decomposition matrices corresponding to the covariance matrix, which are obtained by performing the SVD on the covariance matrix of the first trajectory matrix.

The generating of the second trajectory matrix may include calculating the plurality of second decomposition matrices using the S matrix, the U matrix, and the V^{T} matrix included in the plurality of first decomposition matrices, based on Equation 3 below:

$${H}_{i} = \sqrt{{λ}_{i}} {U}_{i} {V}_{i}^{T} \qquad [ Equation 3 ]$$

Hᵢ is the i^{th} second decomposition matrix of the plurality of second decomposition matrices, λ is the i^{th} eigenvalue of the covariance matrix, Uᵢ is the i^{th} column vector of the U matrix, and

$${V}_{i}^{T}$$

is the i^{th} row vector of the V matrix.

The generating of the second trajectory matrix may include selecting some matrices from among the plurality of second decomposition matrices by a preset number in order of the magnitude of the cardiac signal, filtering the matrices other than a preset frequency band such that the cardiac signal corresponding to the matrices satisfies the frequency band, and generating the second trajectory matrix based on the remaining matrix remaining after filtering among the matrices.

The extracting of the cardiac signal may further include reconstructing the cardiac signal extracted from the second trajectory matrix based on Equation 4 below:

$$\hat{x} ( k ) = \{ \begin{matrix}{\sum }_{i = 1}^{k} \frac{1}{k} {\hat{x}}_{i , k + 1 - i} & ( 1 \leq k < m ) \\ {\sum }_{i = 1}^{m} \frac{1}{m} {\hat{x}}_{i , k + 1 - i} & ( m \leq k < n ) \\ {\sum }_{i = k - m + 1}^{m} \frac{1}{m + n - k} {\hat{x}}_{i , k + 1 - i} & ( n \leq k \leq m + n - 1 )\end{matrix} \qquad [ Equation 4 ]$$

\hat{x}(k) is the reconstructed k^{th} cardiac signal, k is the number of cardiac signals, and m and n are the number of rows or columns of the trajectory matrix.

The classifying of the subject may include classifying the subject as an arrhythmia patient if the R peak interval in the cardiac signal is greater than or equal to a preset value.

### Effects of the Invention

The disclosed embodiments may provide a non-contact arrhythmia detection method by estimating an electrocardiogram signal from ultra-wideband radar signals using a trajectory matrix.

The disclosed embodiments may provide a non-contact arrhythmia detection method that follows a traditional arrhythmia diagnostic approach by determining the R peak interval in the estimated electrocardiogram signal.

### BRIEF DESCRIPTION OF THE DRAWINGS

FIG. 1 is a block diagram of an arrhythmia detection device according to an embodiment.

FIG. 2 is a diagram illustrating an output of the arrhythmia detection device according to an embodiment.

FIG. 3 is a graph illustrating the performance of the arrhythmia detection device according to an embodiment.

FIG. 4 is a flowchart of an arrhythmia detection method according to an embodiment.

### BEST MODE FOR CARRYING OUT THE INVENTION

Hereinafter, a specific embodiment will be described with reference to the drawings. The following detailed description is provided to facilitate a comprehensive understanding of sensors described herein. However, this is merely an example, and the present invention is not limited thereto.

In describing the embodiments, detailed descriptions of well-known technologies related to the present invention will be omitted if it is determined that the detailed descriptions may unnecessarily obscure the subject matter of the embodiments. In addition, numbers (e.g., first, second, and the like) used in the description of the embodiments are merely identification symbols for distinguishing one component from another component.

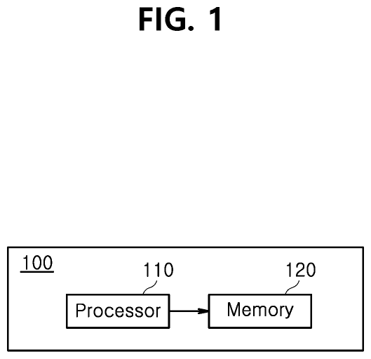

FIG. 1 is a block diagram of an arrhythmia detection device 100 according to an embodiment.

Referring to FIG. 1, the arrhythmia detection device 100 according to an embodiment includes a processor 110 and a memory 120.

The processor 110 obtains raw data of ultra-wideband radar signals of a certain period reflected from a subject.

The ultra-wideband radar is a next-generation sensor that recognizes the surrounding environment by transmitting an impulse signal from a transmission antenna and receiving a signal back from a reception antenna after the transmitted impulse signal is reflected by the subject.

That is, the processor 110 may obtain the raw data of the ultra-wideband radar signals in units of impulses transmitted by the ultra-wide band radar and reflected by the subject. In this case, the raw data may be composed of a frame signal including the signal strength.

The processor 110 may identify the position of the subject's chest wall based on a maximum dynamic variation detected in the raw data.

The processor 110 may generate a first trajectory matrix based on time series data converted from the raw data.

The time series data may be data converted from the raw data related to the position of the subject's chest wall. From this, the processor 110 may extract a cardiac signal by filtering noise unrelated to the cardiac signal (e.g., breathing of the subject or noise from the external environment) from the raw data.

The processor 110 may generate the first trajectory matrix by arranging the time series data converted from the raw data as matrix components.

Specifically, the processor 110 may generate the first trajectory matrix by arranging the time series data converted from the raw data such that components of the remaining diagonals perpendicular to the main diagonal of the matrix are all the same.

For example, the processor 110 may arrange the time series data based on Equation 1 below such that the components of the remaining diagonals perpendicular to the main diagonal of the first trajectory matrix are all the same:

$$H = ( \begin{matrix}{x}_{1} & {x}_{2} & ⋯ & {x}_{n} \\ {x}_{2} & {x}_{3} & ⋯ & {x}_{n + 1} \\ ⋮ & ⋮ & ⋱ & ⋮ \\ {x}_{m} & {x}_{m - 1} & ⋯ & {x}_{L}\end{matrix} ) \qquad [ Equation 1 ]$$

H is the first trajectory matrix, xₐ is the a^{th} time series data, L is the length of the time series data, n and m are arbitrary natural numbers, provided that n=L−m+1, and 2≤m≤n, where m is preferably a value of [L/2].

The processor 110 generates a second trajectory matrix based on the first trajectory matrix.

The processor 110 may generate the second trajectory matrix based on a covariance matrix of the first trajectory matrix.

Preferentially, the processor 110 may calculate the covariance matrix of the first trajectory matrix based on the Equation 2 below:

$$C = \frac{1}{n} {HH}^{7} \qquad [ Equation 2 ]$$

C is the covariance matrix, H is the first trajectory matrix, H^{T} is the transpose of the first trajectory matrix, and n is the number of the time series data.

The processor 110 may perform the SVD on the covariance matrix of the first trajectory matrix to generate the second trajectory matrix.

The processor 110 may generate the second trajectory matrix based on a plurality of first decomposition matrices calculated by performing the SVD on the covariance matrix of the first trajectory matrix.

Specifically, the processor 110 may calculate, as the plurality of first decomposition matrices, at least one of the S matrix, the U matrix, the V^{T} matrix, and the V matrix.

The S matrix is a diagonal matrix where eigenvalues (λ₁, λ₂, . . . , λₘ) of the covariance matrix are arranged as the main diagonal. The eigenvalues may be sorted in descending order.

The U matrix is a left singular matrix, where each column vector can contain an orthogonal vector that captures a pattern and a correlation in the raw data. In this case, the U matrix may include a normalized orthogonal eigenvector corresponding to the S matrix.

The V^{T} matrix may include, as a transpose of a right singular matrix, a right singular vector that is an orthogonal vector describing the relationship of the raw data.

The processor 110 may calculate a plurality of second decomposition matrices that, when combined, become the first decomposition matrix as a decomposition matrix of the first trajectory matrix, based on the plurality of first decomposition matrices corresponding to the covariance matrix.

In other words, the processor 110 may decompose the first trajectory matrix into the plurality of second decomposition matrices by using the plurality of first decomposition matrices.

Specifically, the processor 110 may decompose the first trajectory matrix into the plurality of second decomposition matrices based on the Equation 3 below:

$${H}_{i} = \sqrt{{λ}_{i}} {U}_{i} {V}_{i}^{T} \qquad [ Equation 3 ]$$

Hᵢ is the i^{th} second decomposition matrix of the plurality of second decomposition matrices, λ is the i^{th} eigenvalue of the covariance matrix, Uᵢ is the i^{th} column vector of the U matrix, and

$${V}_{i}^{T}$$

is the i^{th} row vector of the V matrix.

The processor 110 generates the second trajectory matrix based on the first trajectory matrix.

The processor 110 may generate the second trajectory matrix as the decomposition matrix of the first trajectory matrix, i.e., based on the plurality of second decomposition matrices.

Specifically, the processor 110 may generate the second trajectory matrix based on some matrices selected from among the plurality of second decomposition matrices.

The processor 110 may select some matrices from among the plurality of second decomposition matrices by a preset number, e.g., in order of the magnitude of the cardiac signals.

Thereafter, the processor 110 may filter some matrices so that cardiac signals corresponding to the matrices satisfy a preset frequency band.

Preferably, the frequency band may include a range of 0.7 Hz to 3 Hz, which is the frequency of the common heartbeat.

Accordingly, the processor 110 may remove noise, an outlier, and the like other than the heart frequency to improve the accuracy of the extracted cardiac signal.

Thereafter, the processor 110 may generate the second trajectory matrix based on the remaining matrix remaining after filtering among some matrices.

Specifically, the processor 110 may sum all the remaining matrices to generate the second trajectory matrix.

The processor 110 extracts the cardiac signal based on the second trajectory matrix.

For example, the processor 110 may extract the cardiac signal in the frequency domain by applying a Fast Fourier Transformation to the second trajectory matrix.

The processor 110 may reconstruct the extracted cardiac signal based on the Equation 4 below:

$$\hat{x} ( k ) = \{ \begin{matrix}{\sum }_{i = 1}^{k} \frac{1}{k} {\hat{x}}_{i , k + 1 - i} & ( 1 \leq k < m ) \\ {\sum }_{i = 1}^{m} \frac{1}{m} {\hat{x}}_{i , k + 1 - i} & ( m \leq k < n ) \\ {\sum }_{i = k - n + 1}^{m} \frac{1}{m + n - k} {\hat{x}}_{i , k + 1 - i} & ( n \leq k \leq m + n - 1 )\end{matrix} \qquad [ Equation 4 ]$$

\hat{x}(k) is the reconstructed k^{th} cardiac signal, k is the number of cardiac signals, and m and n are the number of rows or columns of the trajectory matrix.

When the specific wavelength interval in the cardiac signal is greater than or equal to the preset value, the processor 110 may classify the subject as an arrhythmia patient.

When the standard deviation of the specific wavelength interval in the cardiac signal is greater than or equal to the preset value, the processor 110 may classify the subject as an arrhythmia patient.

In particular, the processor 110 may classify the subject as an arrhythmia patient when the R peak interval or the standard deviation of the R peak interval in the cardiac signal is greater than or equal to the preset value.

The memory 120 stores one or more instructions executed by the processor 110.

The memory 120 may store various data used by the processor 110. For example, the memory 120 may include software (e.g., input data or output data for a program executed by the processor 110 and/or instructions associated with the program).

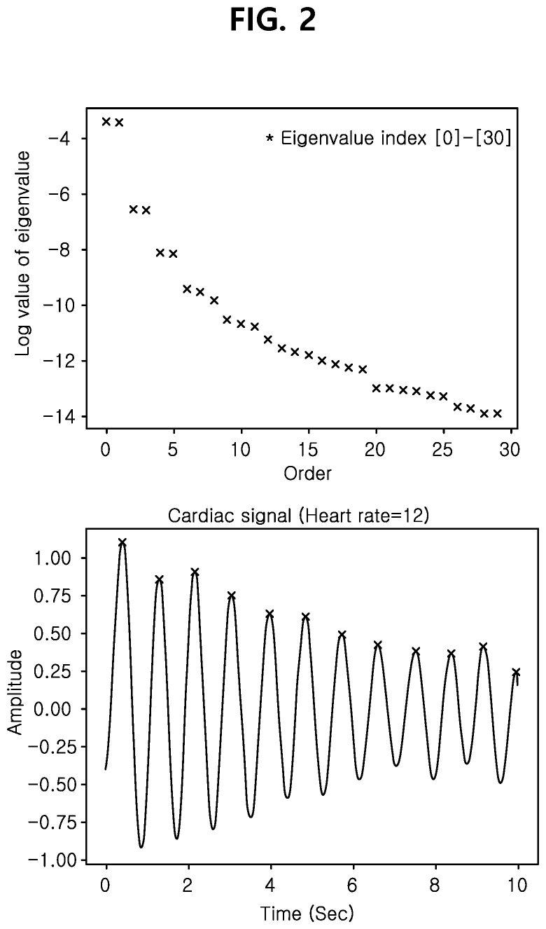

FIG. 2 is a diagram illustrating an output of the arrhythmia detection device 100 according to an embodiment.

Referring to FIG. 2, the upper graph shows log values of eigenvalues of the covariance matrix, and the lower graph shows the cardiac signal corresponding to the upper graph.

As shown in the upper graph, the processor 110 may perform the SVD on the covariance matrix to calculate eigenvalues of the corresponding raw data. At this time, for scaling, the processor 110 may take a logarithm of the eigenvalues to show the distribution of the eigenvalues.

As shown in the lower graph, the processor 110 may extract the cardiac signal based on the second trajectory matrix. The processor 110 may use a wavelet to decompose the frequency component to understand the characteristic of the cardiac signal.

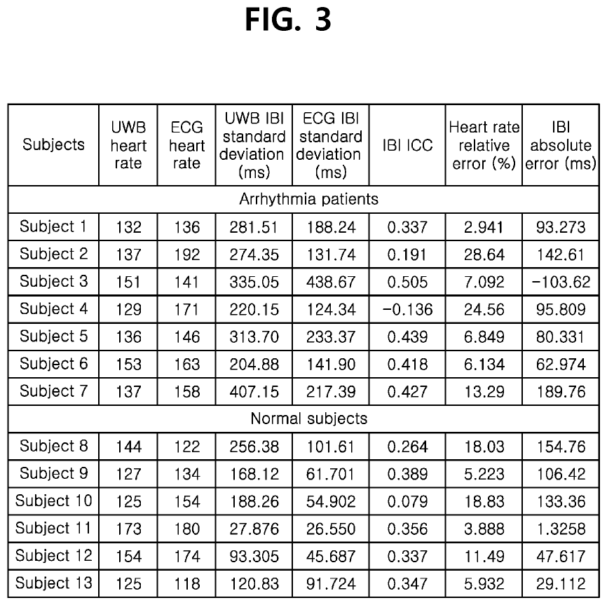

FIG. 3 is a graph illustrating the performance of the arrhythmia detection device 100 according to an embodiment.

Referring to FIG. 3, the errors between the arrhythmia detection device 100 according to an embodiment and the arrhythmia detection method using the existing electrocardiogram signal are compared.

With respect to the heart rate, the arrhythmia detection device 100 according to an embodiment exhibits a matching rate of about 90%, compared with the arrhythmia detection method using the existing electrocardiogram signal.

With respect to ICC, the arrhythmia detection device 100 according to an embodiment indicates that there is a correlation of at less than 0.5, based on an inter-beat interval (IBI), compared with the arrhythmia detection method using the existing electrocardiogram signal.

In particular, the arrhythmia detection device 100 according to an embodiment may classify a normal person and an arrhythmia patient based on a feature value for the IBI, similarly to the arrhythmia detection method using the existing electrocardiogram signal.

The normal subject classified according to the arrhythmia detection method using the existing electrocardiogram signal has an IBI standard value of 105 ms or less, and the arrhythmia patient has an IBI standard value of more than 105 ms.

Similarly, the normal subject classified according to the arrhythmia detection device 100, according to an embodiment, has an IBI standard value of less than or equal to 200 ms, and the arrhythmia patient has an IBI standard value of greater than 200 ms.

As described above, the arrhythmia detection device 100 according to an embodiment may classify whether the subject is an arrhythmia patient based on whether the standard deviation of a specific wavelength interval exceeds a specific threshold value (e.g., 200 ms).

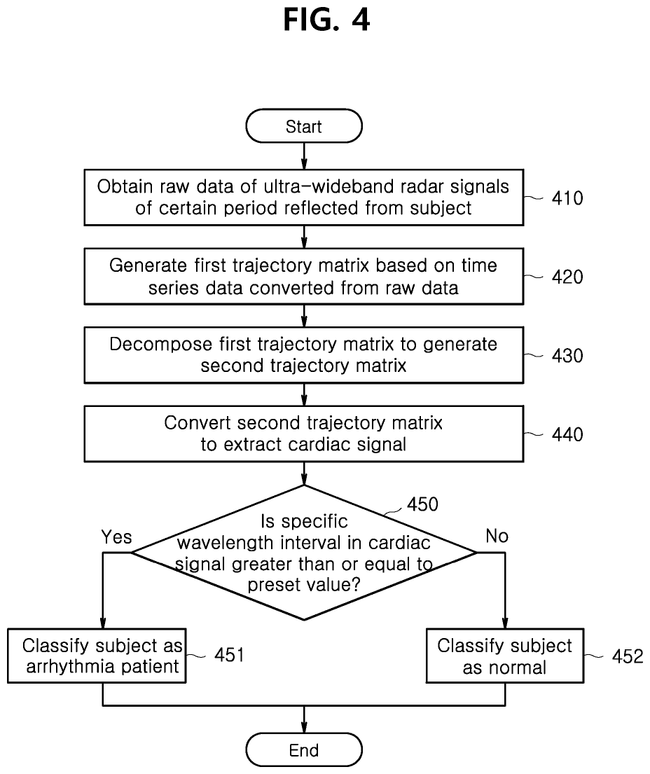

FIG. 4 is a flowchart of the arrhythmia detection method according to an embodiment.

Referring to FIG. 4, the method of FIG. 4 may be performed by the arrhythmia detection device 100 according to an embodiment of FIG. 1.

First, the arrhythmia detection device 100 according to an embodiment obtains raw data of ultra-wideband radar signals of a certain period reflected from a subject 410.

Thereafter, the arrhythmia detection device 100 according to an embodiment generates a first trajectory matrix based on time series data converted from the raw data 420.

Thereafter, the arrhythmia detection device 100 according to an embodiment generates a second trajectory matrix based on the first trajectory matrix 430.

Thereafter, the arrhythmia detection device 100 according to an embodiment extracts a cardiac signal based on the second trajectory matrix 440.

Thereafter, the arrhythmia detection device 100 according to an embodiment determines whether a specific wavelength interval in the cardiac signal is greater than or equal to a preset value 450.

At this time, the arrhythmia detection device 100 according to an embodiment classifies the subject as an arrhythmia patient when the specific wavelength interval in the cardiac signal is equal to or greater than the preset value 451.

On the other hand, when the specific wavelength interval in the cardiac signal is less than the preset value, the arrhythmia detection device 100 according to an embodiment classifies the subject as an arrhythmia patient 452.

Although the method is described as being divided into a plurality of steps in FIG. 4, at least some of the steps may be performed in a different order, may be combined with other steps and performed together, may be omitted, may be divided into detailed steps, or may be performed by adding one or more steps that are not illustrated.

Embodiments of the present invention may include a program for performing the methods described herein on a computer, and a computer-readable recording medium including the program. The computer-readable recording medium may include program instructions, local data files, local data structures, and the like, alone or in combination. The medium may be specially designed and constructed for the present invention or may be commonly used in the field of computer software. Examples of the computer-readable recording medium include a magnetic medium, such as a hard disk, a floppy disk, and a magnetic tape, an optical recording medium, such as CD-ROM and DVD, and a hardware device specifically configured to store and perform program instructions, such as ROM, RAM, flash memory, and the like. Examples of the program may include machine language codes, such as those made by a compiler, as well as high-level language codes that can be executed by the computer using an interpreter or the like.

While exemplary embodiments of the invention have been described in detail above, it will be appreciated by those skilled in the art that various modifications can be made to the above-described embodiments without departing from the scope of the invention. Therefore, the scope of the present invention should not be limited to the described embodiments but should be defined by the following claims and their equivalents.

The terms described below, which are defined in consideration of functions in the present invention, may vary according to an operator's intention, a practice, or the like. Therefore, the definition thereof should be based on the contents throughout the specification. The terms used herein are for the purpose of describing one embodiment only and should in no way be limiting. The singular expressions include plural expressions unless the context clearly dictates otherwise. In the present description, expressions, such as “comprising” or “including,” are intended to indicate certain components, numbers, steps, operations, elements, parts or combinations thereof, and are not interpreted to exclude the presence or possibility of one or more other components, numbers, step, operations, elements, parts, or combinations thereof other than those described.

Furthermore, embodiments described herein may have aspects that are wholly in hardware, partly in hardware and partly in software, or wholly in software. A unit herein refers to hardware, a combination of hardware and software, or a computer-related entity, such as software.

### INDUSTRIAL APPLICABILITY

The radar-based non-contact arrhythmia detection method and device according to an embodiment are available to the digital medical industry by estimating the electrocardiogram signal from ultra-wideband radar signals using the trajectory matrix.

1. An arrhythmia detection device comprising:
one or more processors; and
a memory storing one or more programs executed by the one or more processors, wherein the one or more processors are configured to
obtain raw data of ultra-wideband radar signals of a certain period reflected from a subject,
generate a first trajectory matrix based on time series data converted from the raw data,
decompose the first trajectory matrix to generate a second trajectory matrix,
convert the second trajectory matrix to extract a cardiac signal, and
if a specific wavelength interval in the cardiac signal is greater than or equal to a preset value, classify the subject as an arrhythmia patient.

2. The arrhythmia detection device of claim 1, wherein one or more processors are further configured to identify the position of the subject's chest wall based on a maximum dynamic variation included in the raw data.

3. The arrhythmia detection device of claim 1, wherein the one or more processors are further configured to generate the first trajectory matrix by arranging the time series data as components of the first trajectory matrix based on the following Equation 1 so that components of remaining diagonals perpendicular to a main diagonal of the first trajectory matrix are all the same:
$$H = ( \begin{matrix}{x}_{1} & {x}_{2} & ⋯ & {x}_{n} \\ {x}_{2} & {x}_{3} & ⋯ & {x}_{n + 1} \\ ⋮ & ⋮ & ⋱ & ⋮ \\ {x}_{m} & {x}_{m - 1} & ⋯ & {x}_{L}\end{matrix} ) \qquad [ Equation 1 ]$$
H is the first trajectory matrix, xₐ is the a^{th} time series data, L is the length of the time series data, n and m are arbitrary natural numbers, provided that n=L−m+1, and 2≤m≤n.

4. The arrhythmia detection device of claim 1, wherein the one or more processors are further configured to calculate a covariance matrix of the first trajectory matrix based on the following Equation 2:
$$C = \frac{1}{n} {HH}^{7} \qquad [ Equation 2 ]$$
C is the covariance matrix, H is the first trajectory matrix, H^{T} is the transpose of the first trajectory matrix, and n is the number of the time series data.

5. The arrhythmia detection device of claim 1, wherein the one or more processors are further configured to decompose the first trajectory matrix into a plurality of second decomposition matrices based on a plurality of first decomposition matrices corresponding to the covariance matrix, which are calculated by performing singular value decomposition (SVD) on the covariance matrix of the first trajectory matrix.

6. The arrhythmia detection device of claim 5, wherein the one or more processors are further configured to calculate the plurality of second decomposition matrices using an S matrix, a U matrix, and ta V^{T} matrix included in the plurality of first decomposition matrices based on the following Equation 3:
$${H}_{i} = \sqrt{{λ}_{i}} {U}_{i} {V}_{i}^{T} \qquad [ Equation 3 ]$$
Hᵢ is the i^{th} second decomposition matrix of the plurality of second decomposition matrices, λ is the i^{th} eigenvalue of the covariance matrix, Uᵢ is the i^{th} column vector of the U matrix, and
$${V}_{i}^{T}$$
is the i^{th} row vector of the V matrix.

7. The arrhythmia detection device of claim 5, wherein the one or more processors are further configured to select some matrices from among the plurality of second decomposition matrices by a preset number in order of the magnitude of the cardiac signal, filter the matrices other than a preset frequency band such that the cardiac signal corresponding to the matrices satisfies the frequency band, and generate the second trajectory matrix based on the remaining matrix remaining after filtering among the matrices.

8. The arrhythmia detection device of claim 1, wherein the one or more processors are further configured to reconstruct the cardiac signal extracted from the second trajectory matrix based on the following Equation 4:
$$\hat{x} ( k ) = \{ \begin{matrix}{\sum }_{i = 1}^{k} \frac{1}{k} {\hat{x}}_{i , k + 1 - i} & ( 1 \leq k < m ) \\ {\sum }_{i = 1}^{m} \frac{1}{m} {\hat{x}}_{i , k + 1 - i} & ( m \leq k < n ) \\ {\sum }_{i = k - n + 1}^{m} \frac{1}{m + n - k} {\hat{x}}_{i , k + 1 - i} & ( n \leq k \leq m + n - 1 )\end{matrix} \qquad [ Equation 4 ]$$
\hat{x}(k) is the reconstructed k^{th} cardiac signal, k is the number of cardiac signals, and m and n are the number of rows or columns of the trajectory matrix.

9. The arrhythmia detection device of claim 1, wherein the one or more processors are further configured to, if an R peak interval in the cardiac signal is greater than or equal to a preset value, classify the subject as an arrhythmia patient.

10. An arrhythmia detection method, performed by an arrhythmia detection device comprising one or more processors and a memory storing one or more programs to be executed by the one or more processors, wherein the method comprises:
obtaining raw data of ultra-wideband radar signals of a certain period reflected from a subject;
generating a first trajectory matrix based on time series data transformed from the raw data;
decomposing the first trajectory matrix to generate a second trajectory matrix;
converting the second trajectory matrix to extract a cardiac signal; and
if a specific wavelength interval in the cardiac signal is greater than or equal to a preset value, classifying the subject as an arrhythmia patient.

11. The arrhythmia detection method of claim 10, further comprising identifying the position of the subject's chest wall based on a maximum dynamic variation included in the raw data.

12. The arrhythmia detection method of claim 10, wherein the generating of the first trajectory matrix comprises generating the first trajectory matrix by arranging the time series data as components of the first trajectory matrix based on the following Equation 1 so that components of remaining diagonals perpendicular to a main diagonal of the first trajectory matrix are all the same:
$$H = ( \begin{matrix}{x}_{1} & {x}_{2} & ⋯ & {x}_{n} \\ {x}_{2} & {x}_{3} & ⋯ & {x}_{n + 1} \\ ⋮ & ⋮ & ⋱ & ⋮ \\ {x}_{m} & {x}_{m - 1} & ⋯ & {x}_{L}\end{matrix} ) \qquad [ Equation 1 ]$$
H is the first trajectory matrix, xₐ is the a^{th} time series data, L is the length of the time series data, n and m are arbitrary natural numbers, provided that n=L−m+1, and 2≤m≤n.

13. The arrhythmia detection method of claim 10, wherein the generating of the second trajectory matrix comprises calculating a covariance matrix of the first trajectory matrix based on the following Equation 2:
$$C = \frac{1}{n} {HH}^{7} \qquad [ Equation 2 ]$$
C is the covariance matrix, H is the first trajectory matrix, H^{T} is the transpose of the first trajectory matrix, and n is the number of the time series data.

14. The arrhythmia detection method of claim 10, wherein the generating of the second trajectory matrix comprises decomposing the first trajectory matrix into a plurality of second decomposition matrices based on a plurality of first decomposition matrices corresponding to the covariance matrix, which are calculated by performing singular value decomposition (SVD) on the covariance matrix of the first trajectory matrix.

15. The arrhythmia detection method of claim 14, wherein the generating of the second trajectory matrix comprises calculating the plurality of second decomposition matrices using an S matrix, a U matrix, and a V^{T} matrix included in the plurality of first decomposition matrices based on the following Equation 3:
$${H}_{i} = \sqrt{{λ}_{i}} {U}_{i} {V}_{i}^{T} \qquad [ Equation 3 ]$$
Hᵢ is the i^{th} second decomposition matrix of the plurality of second decomposition matrices, λ is the i^{th} eigenvalue of the covariance matrix, Uᵢ is the i^{th} column vector of the U matrix, and
$${V}_{i}^{T}$$
is the i^{th} row vector of the V matrix.

16. The arrhythmia detection method of claim 14, wherein the generating of the second trajectory matrix comprises:
selecting some matrices from among the plurality of second decomposition matrices by a preset number in order of the magnitude of a cardiac signal;
filtering matrices other than a preset frequency band, so that cardiac signals corresponding to the matrices satisfy the frequency band; and
generating the second trajectory matrix based on the remaining matrix remaining after filtering among the matrices.

17. The arrhythmia detection method of claim 10, wherein the extracting of the cardiac signal further comprises reconstructing the cardiac signal extracted from the second trajectory matrix based on Equation 4:
$$\hat{x} ( k ) = \{ \begin{matrix}{\sum }_{i = 1}^{k} \frac{1}{k} {\hat{x}}_{i , k + 1 - i} & ( 1 \leq k < m ) \\ {\sum }_{i = 1}^{m} \frac{1}{m} {\hat{x}}_{i , k + 1 - i} & ( m \leq k < n ) \\ {\sum }_{i = k - n + 1}^{m} \frac{1}{m + n - k} {\hat{x}}_{i , k + 1 - i} & ( n \leq k \leq m + n - 1 )\end{matrix} \qquad [ Equation 4 ]$$
\hat{x}(k) is the reconstructed k^{th} cardiac signal, k is the number of cardiac signals, and m and n are the number of rows or columns of the trajectory matrix.

18. The arrhythmia detection method of claim 10, wherein the classifying of the subject comprises, if an R peak interval in the cardiac signal is greater than or equal to a preset value, classifying the subject as an arrhythmia patient.
